(19) United States
(12) Patent Application Publication
(10) Pub. No.: US 2005/0033200 A1
(43) Pub. Date: Feb. 10, 2005

(54) Human motion identification and measurement system and method

(76) Inventors: Wayne A. Soehren, Wayzata, MN (US); Charles T. Bye, Eden Prairie, MN (US)

Correspondence Address: HONEYWELL INTERNATIONAL INC., Law Dept. AB2, P.O. Box 2245, Morristown, NJ, 07962-9806, US

(21) Appl. No.: 10/634,931

(22) Filed: Aug. 5, 2003

(51) Int. Cl. A61B 5/103; A61B 5/11

(52) U.S. Cl. 600/595

(57) ABSTRACT

A system and method for classifying and measuring human motion senses the motion of the human and the metabolism of the human. A motion classification unit determines the motion type being carried out by the human and provides the motion classification information to an energy estimator and a health monitor. The energy estimator also receives the metabolism information and therefrom provides an estimate of energy expended by the human. The health monitor triggers an alarm if health related thresholds are traversed. The motion classification is also provided to a processing unit that in turn provides the data to a Kalman filter, which has an output that is provided as feedback to the motion classification unit, the energy estimator and health monitor. Altimeter, GPS and magnetic sensors may also be provided for monitoring the human motion, and initial input and landmark input data inputs are provided to the system.

### BACKGROUND OF THE INVENTION

1. Field of the Invention

The present invention relates generally to system and method for measuring human motion, classifying the motion and determining activity level and energy expenditure therefrom.

2. Description of the Related Art

The measurement of human motion is of interest in various fields. For example, the location of a person may be of interest for security purposes. Human motion detection may be used for monitoring persons with health problems so that help can be sent should they fall or otherwise become incapacitated.

The measurement of human motion is disclosed in U.S. Pat. No. 6,522,266. Motion sensors mounted on the human sense the motion and output signals to a motion classifier. A Kalman filter provides corrective feedback to the first position estimate. A GPS can be provided as a position indicator. Position estimates and distance traveled are determined.

In anticipation of the availability of extremely small, low-cost, and low-power inertial measurement units (IMUs) based on MEMS (Micro Electro-Mechanical System) technology, human-motion-based navigation algorithms utilizing gyroscopes, accelerometers, and magnetic sensors to accurately compute the position of personnel are being developed. First-generation human-motion-based navigation algorithms are based on traditional inertial navigation algorithms tuned by a feedback Kalman filter when external aids, such as GPS (Global Positioning Satellite), magnetometer, or other RF (Radio Frequency) ranging measurements are available. An independent measurement of distance traveled is based on human motion models as another aiding measurement to the Kalman filter. This allows the algorithm to combine the features of dead reckoning and inertial navigation, resulting in positioning performance exceeding that achieved with either method alone.

First-generation human-motion-based navigation algorithms have been developed and demonstrated, with good results in terms of low positioning errors.

With MEMS technology it is possible to build navigation systems including a GPS, inertial measurement unit (IMU), and magnetometer in packages small enough to be easily mounted on a belt or small pack and used as a personal navigation system. The GPS or other RF positioning aids help control any navigation error growth. Dead reckoning techniques provide a solution; however, for best performance, these techniques require the person to move in a predictable manner (i.e., nearly constant step size and in a fixed direction relative to body orientation). Unusual motions (relative to walking) such as sidestepping are not handled and can cause significant errors if the unusual motion is used for an extended period of time.

Human-motion-based navigation algorithms incorporate elements of dead reckoning and inertial navigation algorithms while minimizing the hardware required. A typical dead reckoning system consists of a magnetometer (for heading determination) and a step detection sensor, usually an inexpensive accelerometer. If a solid-state, “strap-down” magnetometer (consisting of three flux sensors mounted orthogonally) is used, the dead reckoning system requires a three-axis accelerometer set to resolve the magnetic fields into a heading angle. A typical IMU consists of three gyros and three accelerometers so that by adding a strap-down magnetometer to an IMU, all the sensors required for dead reckoning or strap-down inertial navigation are contained in a single device.

The human-motion-based navigation algorithm has developed techniques to estimate distance traveled independent of traditional inertial sensor computations while allowing the individual to move in a more natural manner, and integration of inertial navigation and the independent estimate of distance traveled to achieve optimal geolocation performance in the absence of GPS or other RF aids.

To estimate the distance traveled by a walking human, count the steps taken and multiply by the average distance per step. An IMU on a walking human results in gyro and accelerometer data showing each step. A generally linear relationship between step size and walking speed is present over various walking speeds, as described in the book Biomechanics and Energetics of Muscular Exercise by Rodolfo Margaria, ch. 3, pp. 107-124, Oxford Clarendon Press, 1976. By algebraic manipulation the step size is expressed in terms of step frequency, which is computed from the step detections. This equation is the basis for the step model used to estimate the distance traveled in the algorithms, which is coupled with a heading measurement from the magnetometer or inertial navigation to form an input suitable for aiding the navigation equations via a Kalman filter.

The human-motion-based navigation algorithm integrates the distance traveled estimate from the step model with inertial navigation. Integration is done via a multi-state Kalman filter, which estimates and feeds back the traditional navigation error corrections as well as step model and magnetometer corrections. In one example, the Kalman filter is a 30 state filter, although of course other values may be used. When GPS or other RF aids are available, the individuals step model is calibrated, along with the alignment of the IMU and magnetometer.

When external RF aids are not available, the performance of the algorithms is very similar to a dead-reckoning-only algorithm. However, Kalman filter residual testing detects poor distance estimates, allowing them to be ignored, thus improving the overall solution. The residual test provides a reasonableness comparison between the solution based on the distance estimate (and heading angle) and the solution computed using the inertial navigation equations. A simple case to visualize is a sidestep. The step model uses the heading as the assumed direction of travel. However, the actual motion was in a direction 90° off from the heading. The inertial navigation algorithms will accurately observe this, since acceleration in the sideways direction would be sensed. The difference in the two solutions is detected by the residual test, and the step model input to the Kalman filter would be ignored.

A technique has been developed, using the heading rate of change from the inertial navigation equations, to “cut out” use of the distance estimate as an aiding source when the rate of change exceeds a specified threshold. This can provide significant benefits to position accuracy.

The first-generation human-motion-based navigation algorithms have been demonstrated using a Honeywell Miniature Flight Management Unit (MFMU), Watson Industries magnetometer/IMU (1-2° heading accuracy), Honeywell BG1237 air pressure transducer, and a Trimble DGPS base station. The key components of the MFMU are a Honeywell HG1700 ring laser gyro (RLG)-based IMU(1°/hr gyro bias, 1 mg accel bias) and a Trimble DGPS-capable Force 5 C/A-code GPS receiver. These components were mounted in a backpack and carried over various terrain. Test runs were preceded by a “calibration” course during which a DGPS was available to calibrate heading and the person's step model. During the demonstration, data were collected and recorded for all sensors in the backpack. The data were then processed offline to determine the results.

The first-generation human-motion-based navigation algorithms blend inertial navigation and dead reckoning techniques to provide a geolocation solution. By adding detection and models for additional motion types, such as walking up stairs, down stairs, and backwards, the performance and robustness of the algorithms can be increased.

In a motion classification project, two groups of sensors were attached on human body: inertial gyroscopes and accelerometers. Each group has 3 sensors which were used to measure the angular accelerations and linear accelerations along X-axis (defined as forward direction perpendicular to human body plane), Y-axis (defined as side-ward direction perpendicular to X-axis) and Z-axis (defined as the direction perpendicular to X and Y axes and by right-hand rule). The digitized (100 samples/second) time-series signals for the six sensors were collected for several typical human motions, including walking forwards, walking backwards, walking sideways, walking up and down a slope, walking up and down stairs, turning left and right and running, etc, with a goal to identify the human motion.

The time-series signals were divided into 2.56-second (which corresponds to 256 data points so efficient FFT computation can be done) long signal segments. Data analysis and the classification were based on the information embedded in each signal segment (Note there were 6 signal slices for 6 sensors in each segment). Features extracted from the signal segment were fed into an SOM (Self-Organizing Map) neural network for clustering analysis as well as classification. In other words, the SOM is used to examine the goodness of the features and to analyze/classify the inputs. Once the features are chosen, other classifiers can also be used to do the classification work.

The steps involved include,

1. Construct samples: Segment the signals for all kinds of different motion patterns (stationary/left turn/right turn/walking flat/walking on slope/walking up and down stairs/etc);

2. Data reduction/feature extraction: Use FFT (Fast Fourier Transform) to transform the original data to frequency domain. Since the information or the energy of the signal are primarily concentrated in low frequency components, the frequency components (coefficients) higher than a cutoff frequency can be thrown away without significant loss of information. Empirical observation also shows that the magnitudes of FFT coefficients with a frequency equal to or greater than 16 Hz are very small. So the cutoff frequency can be set to 15 Hz. By doing this, the number of data points for each sensor can be reduced to 40 from 256 (details see below). The input feature vector can then be formed by keeping the lower 40 frequency coefficients for each sensor. The vector length would be 40*6=240 if data from 6 sensors are put together and be 120 if gyroscope data and acceleration data are used separately (this can avoid input scaling problem). This step is also helpful for suppressing high frequency noise.

3. Clustering: According to step 2, the dimensionality of input space is very high (120 or 240). SOM is a good tool for clustering analysis of high dimensional data. SOM has several good properties: a) it can do clustering automatically by organizing the position of neurons in the input space according to the intrinsic structure of the input data; b) it is robust (tends to produce stable result given fixed initial conditions compared to vector quantization method); c) it is convenient for data visualization.

4. Explanation/visualization of the SOM results: After training, each neuron in the map space corresponds to one feature or one data cluster (it is possible multiple neurons reflect one cluster when the number of neurons is larger than the number of features).

5. Prediction: Given a future input vector, the neuron which has the smallest distance from the input vector in the input space has an associated class (properties) which are used to predict the motion status of the input vector. Classification may be achieved by using other classifiers such as KNN (K-Nearest Neighbors), MLP (Multi-Layer Perceptron), SVM (Support Vector Machine), etc.

### SUMMARY OF THE INVENTION

The present invention provides for sensing and measurement of human motion, classification of the motion, and determination of energy expenditure as a result of the motion. Sensors of various types are provided on the individual to measure not only inertia and distance but also to determine the respiration rate and heart rate of the individual during the activity, as well as hydration level, blood oxygen level, etc.

In a preferred embodiment, a telecommunications apparatus is provided to transmit the sensor information to a remote location for monitoring, recording and/or analysis.

### BRIEF DESCRIPTION OF THE DRAWINGS

FIG. 1 is a schematic representation of a person whose motion is being monitored by the present invention; and

FIG. 2 is a functional block diagram of a system for monitoring human motion according to the principles of the present invention.

### DETAILED DESCRIPTION OF THE PREFERRED EMBODIMENTS

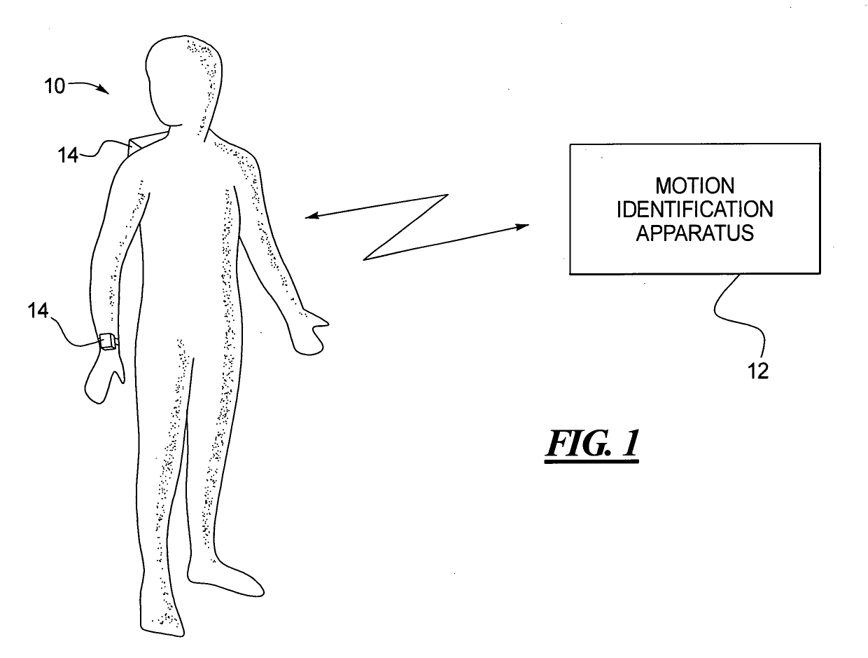

FIG. 1 shows a person 10 whose motion is being monitored by a human motion identification apparatus 12. The person 10 moves about and the motion identification apparatus 12 measures the location of the person 10, the distance moved and a classification of the motion, whether it be standing (no motion), walking (slow motion), or running (fast motion). The positional information may also help to classify the motion as to sitting, standing or laying down, if the person is stationary, or may identify the motion as climbing stairs, for example.

Sensors 14 are attached to the body of the person being monitored. The sensors 14 include inertial gyroscopes and accelerometers, which are preferably mounted on the torso. The sensors 14 are grouped in threes, so that angular and linear motion can be measured in each of the three axes, the X-axis, Y-axis and Z-axis. The digitized time signals for the sensor outputs are collected to determine typical human motions, including walking forwards, walking backwards, walking sideways, walking up and down a slope, walking up and down stairs, turning left and right and running, etc.

In addition, sensors 14 for respiration, pulse and possibly other sensors are attached to the person's body, either on the torso or on one or more limbs. These further sensors monitor the activity level of the person so that determinations can be made about the energy expenditure required for a given amount of movement. The health condition of the person can thereby be monitored.

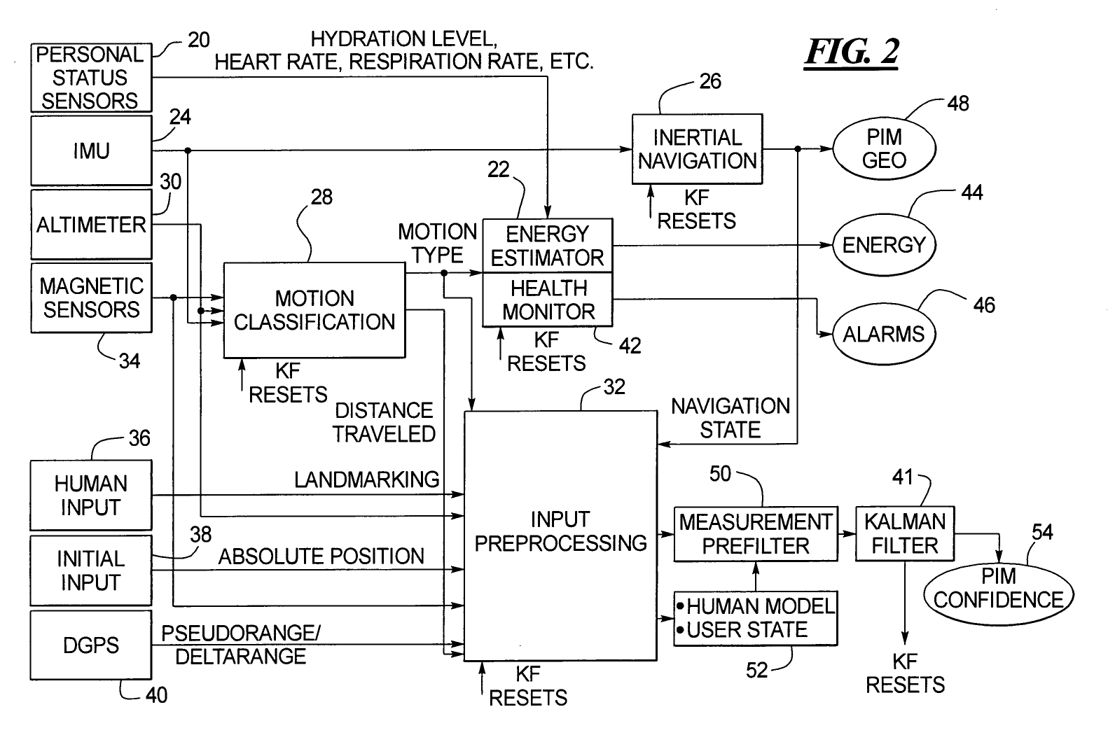

In FIG. 2, the present invention includes a set of personal status sensors 20 to be worn by a person who is being monitored. In one example, the personal status sensors 20 include a hydration level sensor, a heart sensor, a respiration sensor, and perhaps other sensors such as a blood oxygen sensor. For example, the respiration sensor may be an auditory sensor to detect the sounds of breathing. The heart or pulse sensor may be an electrical sensor while the oxygen sensor may be an optical sensor. The hydration sensor may be a capacitance sensor. These sensors detect the metabolism of the person. The output of the personal status sensors is provided to an energy estimating unit 22.

An inertial measurement unit (IMU) 24 is provided which senses the changes in movement of the person being monitored. The inertial measurement sensor unit 24 includes gyroscopic sensors for angular motion and accelerometers for linear motion. The output of the inertial measurement unit 24 is provided to an inertial navigation system 26 and to a motion classification system 28. Further sensors provided on the person being monitored include an altimeter 30, which measures changes in altitude by the person. The altimeter provides its output to the motion classification system 28 and to a input preprocessing unit 32. Magnetic sensors 34 provide direction or heading information and likewise provide its output to the motion classification system 28 and to the input preprocessing unit 32.

The system according to the present invention has inputs in addition to those provided by the sensors of the human motion. For example, a human input 36 is provided for landmarking, the human input 36 being provided to the input preprocessing 32. On example of such a human input 36 is a keyboard and/or pointer device. An initial input unit 38 is provided to set the absolute position of the person being monitored. In addition, a Global Positioning Satellite (GPS) unit or Differential Global Positioning Satellite (DGPS) unit 40 is connected to the input preprocessing unit 32 to provide pseudo-range or delta range information. The DGPS is preferred over the GPS but requires more infrastructure. Either will work in the present application, however.

Among the units which receive input data from the sensors is the above-mentioned motion classification unit 28. The motion classification unit 28 also has an input from a Kalman filter 41 for Kalman filter resets. From these inputs an output is generated to indicate the motion type, which information is transmitted to the energy estimator 22 and health monitor units 42. A further output of the motion classification unit 28 provides information on distance traveled, which information is presented to the input preprocessing unit 32. The motion classification unit 32 may be constructed and operated in accordance with the device disclosed in the U.S. Pat. No. 6,522,266 B1, which is incorporated herein by reference.

The energy estimator unit 22 and health monitor 42 receives the motion type data from the motion classification system, along with the personal status sensor data and a Kalman filter reset data and from this information generates two items of information. First, energy information is provided by the energy estimator 22, which indicates the level of energy expenditure 44 by the person being monitored. This information may be useful in a fitness program, health rehabilitation program—such as post surgery or post injury rehabilitation—or in a weight loss program.

The health monitor 42 provides an output to one or more alarms 46. When the activity level of the person being monitored falls below a predetermined threshold, an alarm 46 is sounded. For example, the alarm 46 may sound to indicate that the person being monitored has fallen, or perhaps they have been stricken with a heart attack, stroke, respiratory disorder, or the like. The alarm 46 may be sounded to a health monitoring service, hospital staff, emergency medical personnel, or other health care provider. The alarm 46 may be sounded to family members or household personnel as well. The alarm is useful to indicate that the person being monitored needs prompt medical attention.

Another aspect of the health monitor determines if some monitored characteristic of the person falls below or rises above a threshold. For example, the breathing rate may increase as the result of a condition, so that the alarm 46 is sounded to indicate the need for attention.

The present monitoring system may be used as a biofeedback system for a person seeking to increase activity to thereby improve health and fitness, so that the alarms 46 may sound to the person being monitored to remind them to increase activity levels. Weight loss goals may be achieved by ensuring that the person maintains a given activity level, for example. Such a reminder system can also be used to remind persons whose jobs or situations require long periods of sitting to get up and walk about so as to reduce the chance of blood clots or other circulation or nerve problems in the lower extremities.

The inertial navigation system 26 which receives data from the inertial measuring unit 24 also received data from the Kalman filter 41. The inertial navigation unit 26 outputs information on the navigation state of the person being monitored to the input preprocessing unit 32 as well as to a Position, Individual Movement unit (PIM) 48. Such a Position, Individual Movement unit 48 may have a geographic function. The PIM unit can also be described as a position, velocity and altitude or orientation unit.

The input preprocessing unit 32 receives the motion type data from the motion classification unit 28, the landmarking data from the human input 36, the altitude information from the altimeter 30, the absolute position information from the initial input unit 38, the magnetic direction information from the magnetic sensors 34, the pseudo-range or delta range information from the Global Positioning Satellite (GPS) system or differential global positioning satellite system (DGPS) 40 and the distance traveled information from the motion classification unit 28, as well as data from the Kalman filter 41. From these inputs, the input preprocessing unit 32 provides data on the measured motion to a measurement pre-filter 50. The measurement pre-filter 50 has provided to it a human motion model 52 and information on the state of the person (the user) being monitored. The output of the measurement unit 50 is provided to the Kalman filter 41, which in turn provides the information to a Position, Individual Motion confidence unit 54. This is an estimate of how well the position, velocity and attitude are known. The Kalman filter provides this as a covariance of each of the navigation states. For position, this is expressed in meters; in other words a position of x, y, and z with an accuracy of n meters. The position information also provides velocity in meters per second and attitude in radians (or other angular measurement). The Kalman filter 41 also generates signals as Kalman filter resets that is provided to the inertial navigation system 26, the energy estimator and health monitor units 22 and 42, the motion classification unit 28 and the input preprocessing unit 32.

The present invention extends the previous motion classification algorithms from measuring the distance a person moved to identifying the type of activity the person is performing. In addition, other sensors in the system identify the energy being expended by the person to perform a task. A core system monitors simple activity history, time activity, activity summary and download information. Components of the system include accelerometers, a processor, data storage, batteries, communications ports including wired ports or IR ports. Further components include gyros and a GPS system to provide activity identification and location information. A respiratory monitor, such as an audio monitor, and a pulse monitor provide estimates of the person's energy expenditure. A cellular telecommunications system enables automated download of the data, real time monitoring and emergency calling capability.

The present invention provides information for motion studies, improving athletic performance, monitoring assembly line workers or other worker motions, determining levels of effort required for tasks, etc.

It is foreseen to sense the human motion by sensors that are remote from the human. For example, it may be possible in some situations to monitor respiration, and motion be sound and motion sensors in a room and so the human would not have to wear the sensors. However, for the most reliable sensing and for mobility of the person, the sensors should be worn on the person's body.

Although other modifications and changes may be suggested by those skilled in the art, it is the intention of the inventors to embody within the patent warranted hereon all changes and modifications as reasonably and properly come within the scope of their contribution to the art.

1. A human motion classification and measurement system, comprising:
sensors for sensing a human;
a motion classification unit connected to receive data from said sensors;
an energy estimator unit connected to receive data from at least one of said motion classification unit and said sensors; and
a Kalman filter connected to receive data from said motion classification unit and from said sensors, said Kalman filter having an output connected to said motion classification unit and said energy estimator unit so that said energy estimator unit is operable to identify an energy expenditure by the human.

2. A human motion classification and measurement system, comprising:
sensors for sensing a human;
an energy estimator unit and a health monitor unit connected to receive data from said sensors; and
a Kalman filter connected to receive data from said sensors and having an output connected to said energy estimator unit and said health monitor unit so that said energy estimator outputs an estimate of energy expended by the human and so that said health monitor outputs an indication of health of the human.

3. A human motion classification and measurement system as claimed in claim 2, further comprising:
an alarm connected to an output of said health monitor unit to indicate traversal of a threshold.

4. A human motion classification and measurement system, comprising:
a personal status sensor for mounting on a human;
motion sensors for mounting on a human;
a motion classification unit connected to receive data from said motion sensors and generate therefrom a motion type indicator signal; and
an output unit connected to said personal status sensors and to receive said motion type indicator signal, said output unit providing an output indicating a status of human activity of the human.

5. A human motion classification and measurement system as claimed in claim 4, wherein said output unit includes an energy estimator unit operable to provide an estimate of energy expended by the human and a health monitor unit operable to activate an alarm upon traversal of a health threshold.

6. A human motion classification and measurement system as claimed in claim 4, wherein said personal status sensor includes at least one of a heart rate sensor and a respiration sensor and a hydration sensor.

7. A human motion classification and measurement system as claimed in claim 4, wherein said motion sensors are inertial sensors including gyroscopic sensors and accelerometers.

8. A human motion classification and measurement system as claimed in claim 4, further comprising:
an altimeter for mounting on the human and having an output connected to said motion classification unit; and
a magnetic sensor for mounting on the human and having an output connected to said motion classification unit.

9. A human motion classification and measurement system as claimed in claim 4, further comprising:
a filter connected to receive data from said motion classification unit, said filter having an output connected to said motion classification unit and to said output unit.

10. A human motion classification and measurement system, comprising:
personal status sensors for mounting to a human;
inertial sensors for mounting to the human;
an altimeter for mounting to the human;
a magnetic sensor for mounting to the human;
a global positioning satellite sensor for mounting to a human;
a motion classification unit having inputs connected to said inertial sensors and said altimeter and said magnetic sensors, said motion classification unit having outputs for data identifying motion type of the human and distance traveled by the human;
an energy estimator and health monitor unit having inputs connected to said personal status sensors and said output of said motion classification unit for motion type data to output energy expenditure information on the human motion and to trigger an alarm upon traversal of a health threshold;
an inertial navigation unit connected to receive data from said inertial sensors and having a navigation state output;
an input preprocessing unit having inputs connected to said global positioning satellite sensor and said magnetic sensor and said altimeter and said motion classification unit and having an output; and
a filter connected to receive data from said output of said input preprocessing unit, said filter having an output connected to said motion classification unit and said energy estimator and health monitor units and said inertial navigation unit.

11. A human motion classification and measurement system as claimed in claim 10, further comprising:
a measurement prefilter connected between said input preprocessing unit and said filter; and
a human model provided as input to said measurement prefilter.

12. A human motion classification and measurement system as claimed in claim 10, further comprising:
an initial input to said input processing unit.

13. A human motion classification and measurement system as claimed in claim 10, further comprising:
a human input to said input preprocessing unit.

14. A human motion classification and measurement system, comprising:
personal status sensors for mounting to a human including a respiration sensor and a heart rate sensor and a hydration sensor;
inertial sensors for mounting to the human including three axis gyros and three axis accelerometers;
an altimeter for mounting to the human;
a magnetic sensor for mounting to the human;
a differential global positioning satellite sensor for mounting to a human;
a motion classification unit having inputs connected to said inertial sensors and said altimeter and said magnetic sensors, said motion classification unit having outputs for data identifying motion type of the human and distance traveled by the human;
an energy estimator and health monitor unit having inputs connected to said personal status sensors and said output of said motion classification unit for motion type data to output energy expenditure information on the human motion and to trigger an alarm upon traversal of a health threshold;
an inertial navigation unit connected to receive data from said inertial sensors and having a navigation state output;
an input preprocessing unit having inputs connected to said global positioning satellite sensor and said magnetic sensor and said altimeter and said motion classification unit and having an output;
a filter connected to receive data from said output of said input preprocessing unit, said filter having an output connected to said motion classification unit and said energy estimator and health monitor units and said inertial navigation unit;
a measurement prefilter connected between said input preprocessing unit and said filter;
a human model provided as input to said measurement prefilter;
an initial input to said input processing unit; and
a human input to said input preprocessing unit.

15. A method for monitoring human motion, comprising the steps of:
sensing motion and metabolism rate of a human;
classifying the motion of the human sensed in said sensing step; and
estimating energy expended by the human from the classified motion and from the metabolism rate.

16. A method as claimed in claim 15, further comprising the step of:
triggering an alarm if a health threshold is traversed.

17. A method as claimed in claim 15, further comprising the steps of:
providing landmarking position data for the human.
